(19) United States
(12) Patent Application Publication
(10) Pub. No.: US 2026/0301159 A1
(43) Pub. Date: Oct. 1, 2026

(54) SYSTEMS AND METHODS FOR AUTOMATED TUMOR SEGMENTATION IN RADIOLOGY IMAGING USING DATA MINED LINE ANNOTATIONS

(71) Applicants: Memorial Sloan-Kettering Cancer Center, New York, NY (US); Memorial Hospital for Cancer and Allied Diseases, New York, NY (US); Sloan-Kettering Institute for Cancer Research, New York, NY (US)

(72) Inventors: Nathaniel Cuthbert Swinburne, New York, NY (US); Vivek Yadav, New York, NY (US)

(21) Appl. No.: 18/998,107

(22) PCT Filed: Jul. 24, 2023
(86) PCT No.: PCT/US2023/070816

§ 371 Date: Jan. 23, 2025

Related U.S. Application Data
(60) Provisional application No. 63/392,007, filed on Jul. 25, 2022.

(51) Int. Cl. G06T 7/00; G06T 7/11; G06T 7/136

(52) U.S. Cl. CPC G06T 7/0012; G06T 7/11; G06T 7/136; G06T 2207/10081; G06T 2207/10088; G06T 2207/20081; G06T 2207/30061; G06T 2207/30096

(57) ABSTRACT

Systems and methods for segmenting pathological features are disclosed. The system can receive a plurality of training images and corresponding data mined or separately introduced line annotations. The system can train a component thereof to generate bounding boxes upon an image of interest based on the training images and the bounding boxes converted from the mined line annotations. The system can detect and refine the segmentations of a pathological feature on the image of interest. The system can reconcile various images, or portions thereof, between the refinement or other processes performed by the system.

### REFERENCE TO RELATED APPLICATIONS

The present application claims priority to U.S. Provisional Patent Application No. 63/392,007, filed Jul. 25, 2022, which is hereby incorporated by reference in its entirety.

### TECHNICAL FIELD

The present application relates generally to detection, quantification or characterization of pathological features based on diagnostic imaging.

### BACKGROUND

Some pathologies can be detectable based on diagnostic images. For example, brain tumors can be visible on one or more magnetic resonance imaging (MRI) images (e.g., across dozens of slices per MRI scan). The pathological lesions, such as tumors, can be characterized based on an annotation. For example, line annotations can be manually entered by a radiologist to define a longitudinal and maximum linear size of a lesion. A burden of a pathological lesion can be related to a volumetric area, surface area, etc. Thus, defining the contours of a lesion (e.g., edges or volumes surrounded by edges), termed “segmentation,” can provide useful information. The definition of such segmentations manually can be time consuming, error prone, and require manual operations by skilled technicians. These efforts present challenges when attempting to quantify or characterize pathological features in diagnostic imaging.

### SUMMARY

The systems and methods of the present disclosure provide techniques for automatically detecting and segmenting pathological lesions in diagnostic images. A first artificial intelligence (AI) model is trained to place bounding boxes over the pathological features. Using an unsupervised segmentation technique, automatically generated segmentation pseudo-masks are created for each detected lesion (bounding box). A second model (or a further model) can perform a self-refinement process to automatically improve the quality of the segmentation pseudo-masks (e.g., by an iterative process comprising retraining a model). The refined images can be validated, such as by a reconciliation process, to determine whether the proposed pseudo-mask update is an improvement over the existing pseudo-mask. The process can result in improved detection or segmentation of pathological features relative to at least some alternative models (e.g., some end-to-end models). Furthermore, this process can, advantageously, avoid or reduce a use of manually segmented training images, which may be time-consuming and laborious to generate. Following training of the tumor segmentation model using this method, a parallel process, such as a variant of the above-mentioned method can be performed to augment the model's performance. A further reconciliation process can reconcile the segmentations provided therefrom. By utilizing the present techniques, segmentations can be formed with an increase of precision or a decrease in a training set data (e.g., resulting in reduced time encoding training data). Therefore, the systems and methods described herein provide improvements to diagnostic imaging technology.

At least one aspect of the present disclosure is directed to a system for automated segmentation of pathological lesions. The system can include one or more processors coupled to a non-transitory memory. The system can receive a plurality of first images. The first portion of the first images can include line annotations corresponding to one or more pathological features. The system can convert line annotations into bounding boxes to train a detection model. The system can define additional bounding boxes for one or more pathological features of the first images or a second image. The system can define a volumetric segmentation corresponding to each bounding box of the second image. The system can iteratively refine the volumetric segmentation based on volumetric segmentation training data to define a refined volumetric segmentation. The system can reconcile the refined volumetric segmentation with another volumetric description corresponding to the pathological feature. The system can generate a presentment image, based on the reconciliation. The system can cause a display of the presentment image via a graphical user interface.

In some implementations, the system can determine a parallel volumetric segmentation. In some implementations, the system can reconcile the refined volumetric segmentation with the parallel volumetric segmentation to define the presentment image. In some implementations, the pathological feature is a tumor and the image is one or more slices of a magnetic resonance imaging (MRI) image. In some implementations, the pathological feature is lung cancer and the image is a computed topography (CT) scan. In some implementations, the plurality of images includes a second portion of the images lacking the line annotations of the pathological features.

At least one aspect of the present disclosure relates to a method for segmenting pathological lesions. The method can be performed, for example, by one or more processors coupled to a non-transitory memory. The method includes receiving a plurality of first images. A first portion of the first images can include line annotations corresponding to one or more pathological features. The method includes converting the line annotations to bounding boxes. The method includes training a bounding box self-labeling component to define a bounding box. The training can be based on the line annotations. The method includes defining additional bounding boxes for one or more pathological features of the first images or a second image. The method includes defining a volumetric segmentation corresponding to each bounding box of the second image. The method includes refining the volumetric segmentation based on volumetric segmentation training data to define a refined volumetric segmentation. The method includes reconciling the refined volumetric segmentation with another volumetric segmentation corresponding to the pathological feature. The method includes generating a presentment image, based on the reconciliation. The method can include causing a display of the presentment image via a graphical user interface.

In some implementations, the method includes determining a parallel volumetric segmentation. In some implementations, the method includes reconciling the refined volumetric segmentation with the parallel volumetric segmentation to define the presentment image. In some implementations the pathological feature is a tumor and the image is one or more slices of a magnetic resonance imaging (MRI) image. In some implementations, the pathological feature is lung cancer and the image is a computed topography (CT) scan. In some implementations, the plurality of images includes a second portion of the images lacking the line annotations of the pathological features.

At least one aspect of the present disclosure relates to a non-transitory computer-readable medium having instructions that, upon execution by a computing device, cause the computing device to perform operations. The operations can include receiving an image comprising a pathological lesion. The operations can include ingesting, by a model trained to detect lesions based on a marking, the image. The operations can include generating, based on the model trained to detect lesions, a marking for one or more unannotated lesions of the image. The operations can include defining a volumetric segmentation corresponding to the marking. The operations can include refining the volumetric segmentation based on volumetric segmentation training data to define a refined volumetric segmentation. The operations can include reconciling the refined volumetric segmentation with another volumetric description corresponding to the one or more unannotated lesions. The operations can include generating a presentment image, based on the reconciliation. The operations can include causing a display of the presentment image via a graphical user interface.

In some implementations, the marking includes a bounding box or a line annotation. In some implementations, the operations include determining a parallel volumetric segmentation. In some implementations, the operations include reconciling the refined volumetric segmentation with the parallel volumetric segmentation to define the presentment image. In some implementations, the unannotated lesions include a tumor and the image is one or more slices of a magnetic resonance imaging (MRI) image. In some implementations, the image includes two-dimensional slices of a three-dimensional structure and the volumetric segmentation is a three-dimensional volumetric segmentation. In some implementations, refining the volumetric segmentation includes iteratively refining the volumetric segmentation based on the volumetric segmentation training data. Each iterative refinement can include generating an iterative segmentation of the pathological lesion.

These and other aspects and implementations are discussed in detail below. The foregoing information and the following detailed description include illustrative examples of various aspects and implementations, and provide an overview or framework for understanding the nature and character of the claimed aspects and implementations. The drawings provide illustration and a further understanding of the various aspects and implementations, and are incorporated in and constitute a part of this specification. It will be readily appreciated that features described in the context of one aspect of the invention can be combined with other aspects. Aspects can be implemented in any convenient form, for example, by appropriate computer programs, which may be carried on appropriate carrier media (computer readable media), which may be tangible carrier media (e.g., disks) or intangible carrier media (e.g., communications signals). Aspects may also be implemented using suitable apparatus, which may take the form of programmable computers running computer programs arranged to implement the aspect.

### BRIEF DESCRIPTION OF THE DRAWINGS

The foregoing and other objects, aspects, features, and advantages of the disclosure will become more apparent and better understood by referring to the following description taken in conjunction with the accompanying drawings, in which:

FIG. 1 depicts an example system for performing pathological feature segmentation to automate detection thereof, in accordance with one or more implementations;

FIG. 2 shows an example of line annotations and a bounding box overlaid over a pathological feature, in accordance with one or more implementations;

FIG. 3 shows segmentation and refinement of a pathological feature defined by a bounding box, in accordance with one or more implementations;

FIG. 4 shows reconciliation between various segmentations, in accordance with one or more implementations;

FIGS. 5A, 5B, and 5C depict example methods for segmenting a pathological feature, in accordance with one or more implementations;

FIGS. 6A, and 6B depict a further example method for segmenting a pathological feature, in accordance with one or more implementations;

FIG. 7 is a block diagram of a server system and a client computer system in accordance with an illustrative embodiment.

### DETAILED DESCRIPTION

The present techniques can define segmentation of pathological features, which can result in determining a burden of a pathological feature with increased granularity or accuracy, or decreased diagnostic time. For example, an election to perform a surgical or non-surgical treatment can be based on a determined burden of the pathological feature. It should be appreciated that various concepts introduced above and discussed in greater detail below may be implemented in any of numerous ways. The disclosed concepts are not limited to any particular manner of implementation. Examples of specific implementations and applications are provided primarily for illustrative purposes. For the purpose of better understanding the present disclosure, a brief overview of the sections of the detailed description may be helpful:

Section A describes systems and methods for performing pathological feature segmentation.

Section B describes a network environment and computing environment which may be useful for practicing various embodiments described herein.

Section C describes an example embodiment, using the systems and methods herein to segment tumors across one or more slices of a brain MRI image.

### A. Automated Tumor Segmentation Using Data Mined Line Annotations

Line annotations are used to define dimensions of tumors or other pathological features such as polyps, cysts, lesions, tissues indicative of stroke, multiple sclerosis, or the like. For example, a first line annotation can define a longest dimension of a pathological feature, and a second line annotation can define a longest perpendicular dimension of the pathological feature. Advantageously, line annotations can be readily tracked, compared, or measured (e.g., between slices of an MRI, longitudinally between patient visits, or laterally between various patients). In some pathological features, such as generally regular oblate ellipsoidal tumors, the line annotations can generally characterize the features of an image. In some pathological features, such as those having irregularly shaped features, the line annotations can less fully characterize the features. For example, two pathological features having similar line annotations can have disparate volumes or other characteristics. For example, a first tumor can have similar line annotations as a second tumor having a greater volume. As used herein, an image can refer to a set of slices of an MRI, MRA, or other 3D scan, to the individual slices or other portions thereof, or to additional diagnostic images such as CT, X-rays, ultrasounds, or the like.

Some images (e.g., MRI images, CT images, X-Ray images, ultrasounds and so forth), or relevant portions thereof, may lack line annotations. For example, a subset of images such as images comprising a maximum dimension of a line annotation, or depicting an adjacent structure to the pathological feature such as an artery or cranial lobe can be selected for marking, and additional images can be unmarked. Moreover, line annotations may not fully characterize various pathological features. However, line annotations can be compared across a plurality of images, patients, or visits to determine a rate of growth, recession, or other change to the pathological feature or another feature of interest, such as to inform treatment decisions.

As noted above, the line annotations may not fully characterize at least some pathological features. Thus, segmentation may be desirable. Segmentation delineates the boundaries of a portion of an image containing a pathological feature, such as by defining one or more edges of the feature, or defining an area of a 2D image or a volume of a 3D image (both of which may be referred to as volumetric, for the sake of brevity since many of the methods disclosed herein are compatible with both two dimensional and three dimensional images). Segmentation can require greater technician time to prepare than line annotations, and can require additional techniques to compare over time, between patients, or the like. Moreover, the segmentation data can include actionable information for a patient, or retrospective information to advance the understanding of the pathological feature. For example, a shape, location, surface roughness, rate of change, or other characteristic of the feature may be associated with a patient outcome, recommended treatment, or be otherwise relevant. Thus, it may be advantageous for a system to automatically segment one or more images of a pathological feature. Such segmentation may be referred to as such, or by reference to a mask (e.g., a mask, a pseudo-mask, a hybrid mask or the like). Such a mask can be an overlay, quantification, pixel map, or other descriptor of the one or more segmentations. Thus, references to the segmentations themselves, or to the masks corresponding to the segmentations can be used interchangeably.

An automated system to determine a first description of the pathological features (such as line annotations or bounding boxes which define a peripheral boundary of the pathological features) may thus be desirable. An automated system to determine a second description (e.g., the segmentation of the pathological feature) may also be desirable. The second description can be defined (at least in part) according to the first description. Thus, a system directed to determining a first description (e.g., bounding boxes) and a second description (e.g., segmentation) may, advantageously, operate with increased performance relative to independent systems. These and other features are described in greater detail herein.

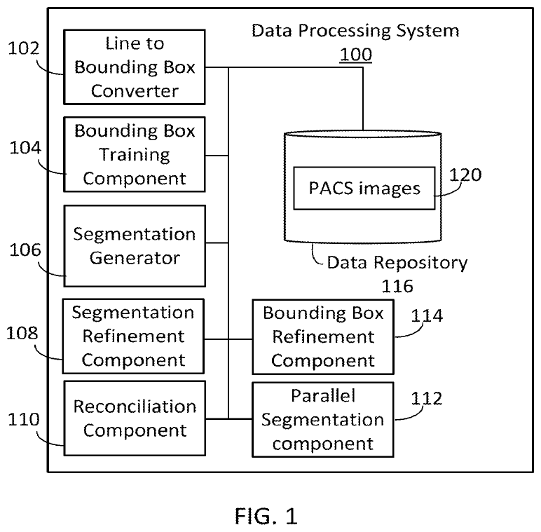

Referring now to FIG. 1, depicted is block diagram of an example data processing system 100, in accordance with one or more implementations. The data processing system 100 can include at least one line to bounding box converter 102. The data processing system 100 can include at least one bounding box training component 104. The data processing system 100 can include at least one segmentation generator 106. The data processing system 100 can include at least one segmentation refinement component 108. The data processing system 100 can include at least one reconciliation component 110. The data processing system 100 can include at least one parallel segmentation component 112. The data processing system 100 can include at least one bounding box refinement component 114. The data processing system 100 can include at least one data repository 116.

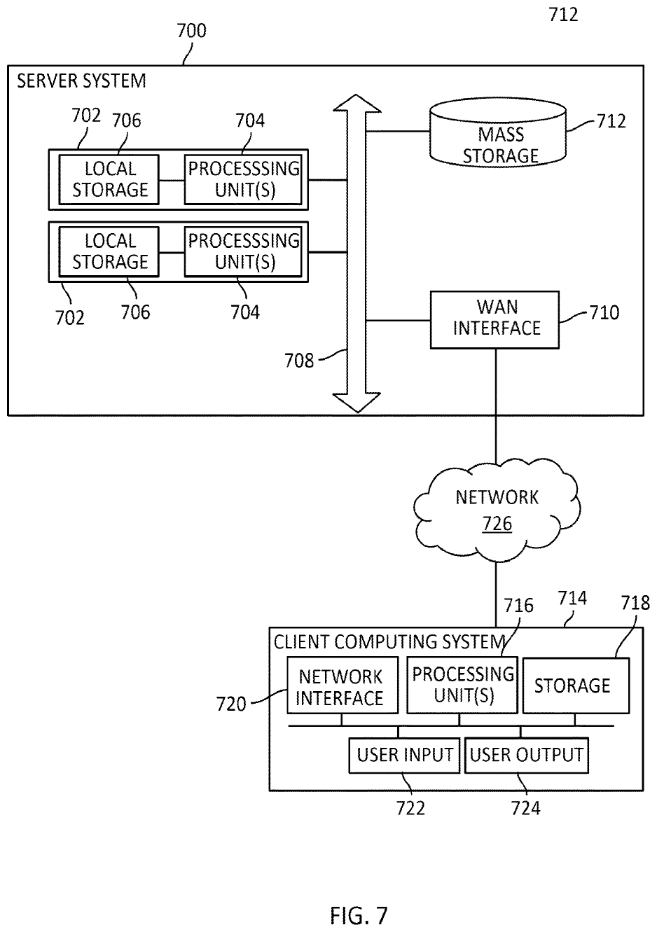

The line to bounding box converter 102, bounding box training component 104, segmentation generator 106, segmentation refinement component 108, reconciliation component 110, or parallel segmentation component 112 can each include a processing unit or other logic device such as programmable logic array engine, or module configured to communicate with the data repository 116 or database. The line to bounding box converter 102, bounding box training component 104, segmentation generator 106, segmentation refinement component 108, reconciliation component 110, or parallel segmentation component 112 can be separate components, a single component, or part of the data processing system 100. The data processing system 100 can include hardware elements, such as one or more processors, logic devices, or circuits. For example, the data processing system 100 can include one or more components, structures or functionality of a computing device depicted in FIG. 7.

The data repository 116 can include one or more local or distributed databases, and can include a database management system. The data repository 116 can include computer data storage or memory and can store a plurality of picture archiving and communications system (PACS) images 120. The PACS images 120 can include a plurality of images, at least a portion of which include a pathological feature. At least a portion of the images including pathological features can include line annotations defining one or more dimensions of the pathological features. For example, the PACS images 120 can include a first line annotation defining a maximum dimension of a tumor and a second line annotation, perpendicular to the first line, the second line defining another maximum dimension of the tumor.

Still referring to FIG. 1, and in further detail, the data processing system 100 can include at least one line to bounding box converter 102 designed, constructed or operational to detect line annotations or define bounding boxes therefrom. For example, the line to bounding box converter 102 can detect one or more line annotations based on metadata associated with an image. For example, the line annotations can be defined within digital imaging and communications in medicine (DICOM) gray scale presentation state (GSPS) files. The line to bounding box converter 102 can extract the line annotations encoded within metadata files and match them to their referenced images using based on a layer, tag, DICOM unique identifiers (UIDs), or other indicia of a computer-edited image. A comment, filename, or other metadata of or associated with an image can include a notice that an image is annotated, such as initials of the annotator or information corresponding to the annotation (e.g., a dimension of a pathological feature). In some embodiments, PACS images 120 can be sourced from a plurality of personnel, departments, or organizations, thus various combinations of the techniques depicted herein can be employed. For example, PACS images 120 sourced from a first organization can include line annotations encoded in GSPS files, whereas PACS images 120 from a second organization may not include colorized line annotations, but may include a file type wherein the image content and annotation thereof are stored as tabular data (e.g., .csv) or distinguishable layers (e.g., .png or .pdf).

The line to bounding box converter 102 can generate a bounding box around a pathological feature. For example, the line to bounding box converter can generate a bounding box defined by two or more corners to encapsulate the line annotations. The bounding box defined by the line to bounding box converter 102 can be square, rectangular, circular, or another shape, such as an irregular shape. In some embodiments, the box can be oriented with reference to an image (e.g., a rectangular box can include upper and lower bounds parallel with the upper and lower bounds of the image, and left and right bounds parallel with the left and right bounds of the image). In some embodiments, the box can be oriented with reference to a pathological feature. In some embodiments, the line to bounding box converter 102 can use edge detection or other techniques, such as the various techniques described herein, to determine a portion of the pathological feature extending beyond the line annotations, such as a salient of an irregularly shaped pathological feature extending beyond a central mass thereof, or to reduce a portion of the bounding box such as due to a concavity of the pathological feature.

The line to bounding box converter 102 can provide training data to train the bounding box training component 104 or to train a model to generate bounding boxes around pathological features. For example, the line to bounding box converter 102 can form bounding boxes for the portion of PACS images 120 having line annotations such as to train a model which can generate bounding boxes for additional pathological features (e.g., those lacking line annotations). In some embodiments, the training data passed to the bounding box training component 104 can include the line annotations. In some embodiments, such as where the line annotations are disposed on another file layer or where a copy of the images lacking the line annotations is available, the training data passed to the bounding box training component 104 can lack the line annotations, which, advantageously, may aid in training the bounding box training component 104 to define bounding boxes based on pathological features rather than line annotations. In some embodiments, the line to bounding box converter 102 and the bounding box training component 104 can share one or more components, memory spaces, or circuits. For example, the line to bounding box converter 102 can be, include, or be included in a training portion of a machine learning model, and the bounding box training component 104 can be include, or be included in a prediction portion of the machine learning model. In some embodiments, the machine learning model can be or include one or more convolutional neural networks (CNNs).

The data processing system 100 can include at least one bounding box training component 104 designed, constructed or operational to train a model to define bounding boxes around pathological features. For example, the bounding box training component 104 can train a model to generate bounding boxes having a defined shape, such as a rectangular or other shape. The bounding box training component 104 can train a model to detect pathological features based on a training set based on line annotations. The bounding box training component 104 can define bounding boxes of various shapes and orientations, as discussed with regard to the line to bounding box converter 102. The bounding box training component 104 can define one or more overlapping bounding boxes. For example, plurality of pathological features can be located in proximity such that the bounding boxes for the respective pathological features overlap, or a single pathological feature can include a plurality of lobes, protractions, or other sub-features which can be separately bounded.

The data processing system 100 can include at least one bounding box refinement component 114 designed, constructed, or operational to refine a model for bounding boxes. For example, the bounding box refinement component can add bounding boxes to various images. For example, the bounding box refinement component 114 can self-label one or more lesions, based on the training of the bounding box training component 104. As used herein, self-labeling refers to the generation of bounding boxes for an image including a pathological lesion which is not defined by line annotations. For example, the bounding box refinement component 114 can receive images lacking line annotations (e.g., because no line annotations are available, or because line annotations are not provided to or accessible by the bounding box refinement component 114). In some embodiment, line annotations may be inaccessible to one or more components based on a selection, such as for a training or demonstration step wherein the bounding boxes are placed based on the pathological image rather than the line annotations. The bounding box refinement component 114 can define one or more bounding boxes according to a detection model, such as a lesion detection model. For example, the bounding box refinement component 114 can receive an expanded training set of images and label the expanded set of training images. The model (e.g., the lesion detection model) can thereafter be refined (e.g., trained) to iterate the model, whereupon a further expanded training set of images can be received, and so on. The bounding box refinement component 114 can iteratively train the model. One or more images received by the bounding box refinement component 114, or otherwise received by the model can be for a patient of interest, such as a patient suspected or known to have a pathological lesion, and wherein a detection or characterization of the pathological lesion can be provided.

The data processing system 100 can include at least one segmentation generator 106 designed, constructed or operational to determined segmentations of a pathological feature bounded or substantially bounded by the bounding boxes. The segmentations can be determined based on various thresholding functions. For example, Otsu's method, a Gaussian model mixture, watershed segmentation, a connected threshold filter, fast marching segmentation, a shape detection filter, an edge detection function, or the like to segment the pathological features can be employed. In some embodiments, the segmentation generator 106 can receive segmentation training data. For example, the segmentation training data can include an edge or threshold segmentation of one or more pathological features, or a pass/fail result from a completed segmentation. In some embodiments, the segmentation generator 106 can determine the segmentations without segmentation training data. For example, the edge detection, segment detection, etc. can determine the location of pathological features. The segmentation generator 106 can determine the segmentations based on the bounding box data alone, or based on additional image data (e.g., to provide a further reference of non-pathological features). In some embodiments, the segmentation generator can be constrained to a single pathological type, such as a tumor, a type of tumor, or a type of tumor in a specified tissue (e.g., a malignant brain tumor). In some embodiments, the segmentation generator can include one or more models directed to various pathological feature types, or a single model configured to segment various pathological feature types.

The data processing system 100 can include at least one segmentation refinement component 108 designed, constructed or operational to refine a determined segmentation. For example, the segmentation refinement component 108 can recursively adjust the segmentation of the pathological features. The segmentation refinement component 108 can perform a fixed number of recursions or a variable number of recursions. For example, the segmentation refinement component 108 can compare a segmentation to a training model to make incremental changes to the segmentation. The segmentation can be a segmentation defined by the segmentation generator or another iteration of the segmentation refinement component 108. For example, the changes can be made along an edge or segment of the image to generate an updated image. The updated image can be passed to a reconciliation component 110 (as is further discussed below) to validate the changes (e.g., the updated image can be a proposed image).

The segmentation refinement component 108 can receive an indication of validation or non-validation from the reconciliation component 110. Responsive to an indication of non-validation, the segmentation refinement component 108 can halt further refinement of one or more segmentations of the image, or refine the image according to alternate criteria (e.g., based on an alternate random seed, resolution, or another refinement model). Responsive to an indication of validation of the proposed refinement, the segmentation refinement component 108 can complete the refinement operation (e.g., if the image meets a criteria for a completion of the refinement process such as a number of cycles or a quantified change from a previous image). Responsive to an indication of validation of the proposed refinement, the segmentation refinement component 108 can re-iterate the image to further refine the segmentation thereof. For example, the various recursions discussed herein can be performed with regard to an image or a feature bounded by a bounding box. For example, an image containing several bounding boxes for respective pathological features can recursively iterate all segmentations of pathological features a fixed number of times, or the various bounding boxes can go through different numbers of refinement operations based on the content thereof.

The data processing system 100 can include at least one reconciliation component 110 designed, constructed or operational to reconcile proposed segmentations. For example, the reconciliation component 110 can determine a completion of a recursive process of the segmentation refinement component 108 in embodiments having a variable number of recursive refinement operations. The reconciliation component 110 can apply various criteria to the images to validate the segmentation updates. For example, the criteria can include fitting the shape to the bounding box. For example, if the shape does not occupy a portion of the bounding box greater than a threshold, the update to the segmentation can be determined invalid. Various thresholds can be defined (e.g., the threshold can be linear in an X direction or a Y direction). For example, the reconciliation component 110 can determine a segmentation which occupies 50% or less of the lateral portion of a bounding box is non-valid. The reconciliation component 110 can apply volumetric thresholds, based on the bounding box or a previous threshold (e.g., 35% of the bounding box can be determined to be occupied by a pathological feature to validate a refinement). The reconciliation component 110 can establish thresholds based on contiguity of a segmentation, such as within a slice or between slices of an MRI image. In some embodiments, one or more criteria can be weighted such that a validation or non-validation is determined based on a combinatorial weighting of the various criteria.

The reconciliation component 110 can reconcile segmentation indications from various systems, methods, or sources. For example, the reconciliation component 110 can reconcile a segmentation defined by the segmentation refinement component 108 with another process, such as a parallel segmentation process, as is discussed with regard to the parallel segmentation component 112, below. The reconciliation of two or more parallel techniques can increase informational diversity and can detect features which may be novel, unusual, or absent from one or more training sets (e.g., pathologies of varying geographic prevalence) which may be undetected or over-detected by one or more techniques.

The data processing system 100 can include at least one parallel segmentation component 112 designed, constructed, or operational to determine segmentation of one or more pathological features. The parallel segmentation component 112 can determine the segmentation based on any technique. In some embodiments the parallel segmentation component 112 can determine the segmentation based on a variant of one or more of the line to bounding box converter 102, bounding box training component 104, segmentation generator 106, segmentation refinement component 108, reconciliation component 110, or bounding box refinement component 114. For example, the data processing system can include a plurality of variants of the systems described above for reconciliation there-between. In some embodiments, the parallel segmentation component 112 can determine segmentation according to another technique, such as a defined detection algorithm, or an end-to-end machine learning model.

In some embodiments, an end-to-end machine learning model can be trained based on one or more bounding boxes or segmentations generated by the bounding box training component 104, segmentation generator 106, or segmentation refinement component 108. In some embodiments, the end-to-end machine learning model can be based on manually segmented images. In some embodiments, parallel segmentation component 112 can be an end-to-end segmentation model which includes different techniques, components, or training data than the other components of the data processing system 100. Advantageously, such a variance can result in informational diversity between the models to reduce duplicate errors between the parallel segmentation component 112 and the other components of the data processing system 100. In some embodiments, the end-to-end segmentation model can be or include same or similar elements of the data processing system. Advantageously, such systems may reduce a complexity of the data processing system and improve interoperability between components.

One or more of the components can be employed for continuous training of the detection or segmentation models such as by continuously data mining the PACS database to extract newly generated line annotations created by radiologists during image interpretation and mark-up. For example, as new diagnostic imaging (e.g., MRI scans) are acquired, the line annotations created by radiologists during image interpretation can be extracted and added to the line annotation training dataset. The system components can process this newly expanded line annotation dataset to generate new bounding boxes and segmentations, improving model performance. In some embodiments, such continuous learning/retraining can occur incident to manual intervention or screening of the disparity between the models. In some embodiments, such continuous learning/retraining can occur automatically (e.g., based on a predicted relative false positive/false negative ratio between models).

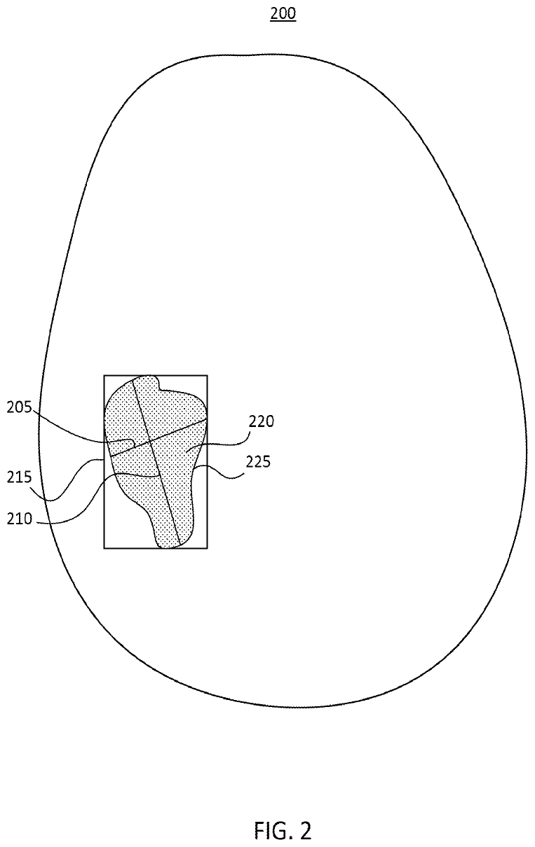

Referring to FIG. 2, an example of line annotations 205, 210 and a bounding box 215 overlaid over a pathological feature is disclosed, in accordance with one or more implementations. In the depicted image, the pathological feature is a brain tumor 220 on a slice of an MRI image 200. Additional slices, images, or the like can include further bounding boxes 215, line annotations, 205, 210, etc. (e.g., of the depicted brain tumor 220 or another pathological feature). A first line annotation 205 can define a first dimension of the brain tumor 220. For example, the first dimension can be a lengthwise dimension of the brain tumor 220 (e.g., a maximum dimension of the brain tumor 220, a maximum dimension of a major feature of the brain tumor 220, a dimension of the brain tumor 220 aligned to a defined direction, such as a predefined direction, or a direction relative to a line annotation of another image). A second line annotation 210 defines another dimension of the brain tumor 220. The second line annotation 210 can be disposed perpendicular to the first line annotation 205. The second line annotation 210 can define a maximum dimension of the brain tumor 220, be disposed medially along the distance of the first line annotation, etc.

A bounding box 215 can be defined around a periphery of the brain tumor 220 or the line annotations. For example, the bounding box 215 can be designed based on the position of the line annotations 205, 210, such as to surround the line annotations 205, 210, and can include an additional margin or offset therefrom. The bounding box 215 can be based on the brain tumor 220. For example, an edge 225 of the tumor can be recognized, and the bounding box can be defined to surround the edge. In some embodiments, no line annotations are present. For example, the bounding box training component can be trained, at least in part, based on line annotations, to detect pathological features such that the pathological features can be detected absent the line annotations. In some embodiments, a plurality of bounding boxes 215 can be assigned to one image (e.g., an image having two similar or dissimilar pathological features). In some embodiments, a plurality of bounding boxes 215 can be assigned to one pathological feature. For example, each pole of a bipolar brain tumor 220 can be surrounded by a bounding box 215.

The bounding box 215 can be located by a machine learning algorithm trained using bounding boxes generated from line annotations 205, 210. For example, a plurality of images comprising a first portion including line annotations and a second portion lacking line annotations can be ingested by a machine learning algorithm. The output of the machine learning algorithm can detect bounding boxes of a pathological feature based on the ingested images. In some embodiments, the machine learning algorithm can include an intermediate operation of forming line annotations or bounding boxes on the portion of images lacking line annotations. For example, the line annotations can generate additional sample data or material for review of the machine learning algorithm.

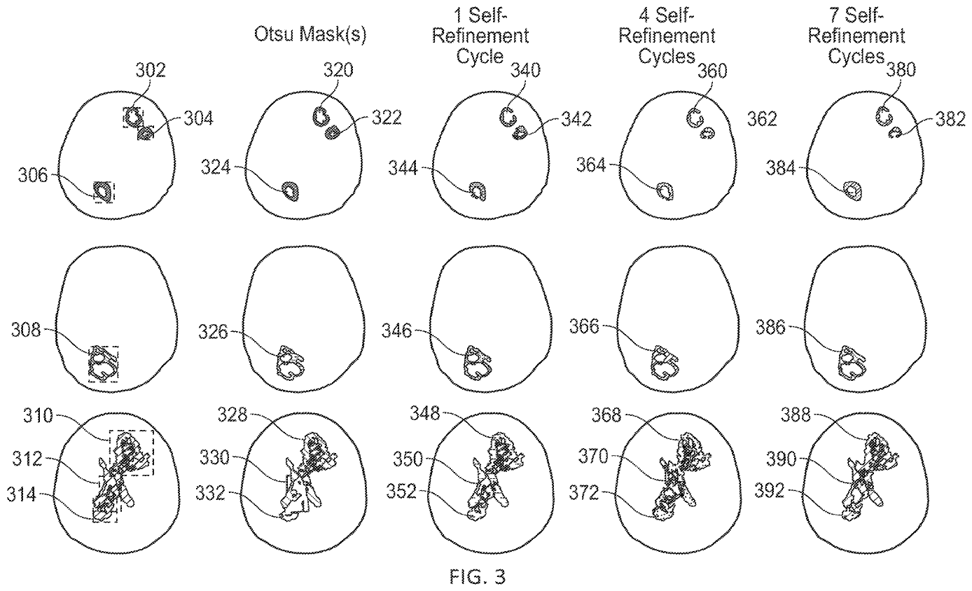

Referring to FIG. 3, segmentation and refinement of a pathological feature defined by a bounding box is depicted, in accordance with one or more implementations. A first row of images includes an image having a first bounding box 302, a second bounding box 304 abutting the first bounding box 302, and a third bounding box 306. The second bounding box and third bounding box can be defined to avoid or ensure an overlap. For example, an avoidance of an overlap can aid each pathological structure of the associated bounding boxes to be atomically processed. An overlap may cause a linkage to be established between the one or more pathological features of the respective bounding boxes. A third bounding box defines a further pathological feature. A fourth bounding box 308 defines one or more pathological features of another image. A fifth bounding box 310, sixth bounding box 312, and seventh bounding box 314 are overlaid on a further image still. The sixth bounding box 312, and seventh bounding box 314 overlap. The fifth bounding box abuts the fourth bounding box.

Edges or bodies of various pathological features can be defined based on various edge detection or threshold functions. For example, Otsu's method can be employed. The edges or bodies of the pathological features can be termed masks, pseudo-masks (e.g., to refer to a computer defined mask, which may also be referred to as a mask, segmentation, contour, or the like), etc. For example, the masks can be or include a detected portion (e.g., the depicted masks of mask 320, 322, 324, 326, 328, 330, or 332). The masks can include or exclude portions surrounded by edges (e.g., internal portion of the pathological feature or laterally surrounded portions of healthy tissue). As depicted, a mask 328 corresponding to the fifth bounding box 310 abuts the mask 330 corresponding to the sixth bounding box 312, which, in turn, overlaps a mask 332 corresponding to the seventh bounding box 314.

The disclosed systems and methods can refine the masks. For example, an automated self-refinement process can include retraining a model (e.g., a CNN) based on the updated mask. A first refinement operation can be based on a baseline segmentation model. A reconciliation process (e.g., to reconcile a refined mask with a previous mask) can be performed to determine an acceptability of a refined mask. For example, a refined mask can be compared to a baseline or a previous mask to determine a difference. For example, if a total volume, edge length, edge location, etc., is adjusted greater than a threshold, relative to a previous mask or a bounding box associated therewith, a refinement can be rejected. As for the other systems and methods disclosed herein, the refinement can be performed on a bounding box basis, an image basis, or another basis, such as a longitudinal series of images of a patient. For example, the refinement of masks 350 and 352 can be based on individual bounding box analysis of each overlapping bounding box 312, 314, image analysis, combined analysis, etc. In some embodiments, a sub-operation to harmonize the overlapping, adjacent or proximal bounding boxes, or images from adjacent images or subsequent visits may be performed.

A refined mask can be further refined by an iterative process, such as by retraining a model, based on the reconciliation process (e.g., by encoding the rejected refinements as a fail condition, or the non-rejected refinements as a pass condition). The process can be iterated to reach a threshold such as a pre-defined number, a characterization of the mask, or another feedback process. For example, a first plurality of masks, 340 through 352 are depicted following a first refinement iteration, a second plurality of masks, 360 through 372 are depicted following a fourth refinement iteration, and a third plurality of masks, 380 through 392 are depicted following a seventh refinement cycle, each plurality of masks corresponding to the first bounding box 302 through the seventh bounding box 314, respectively.

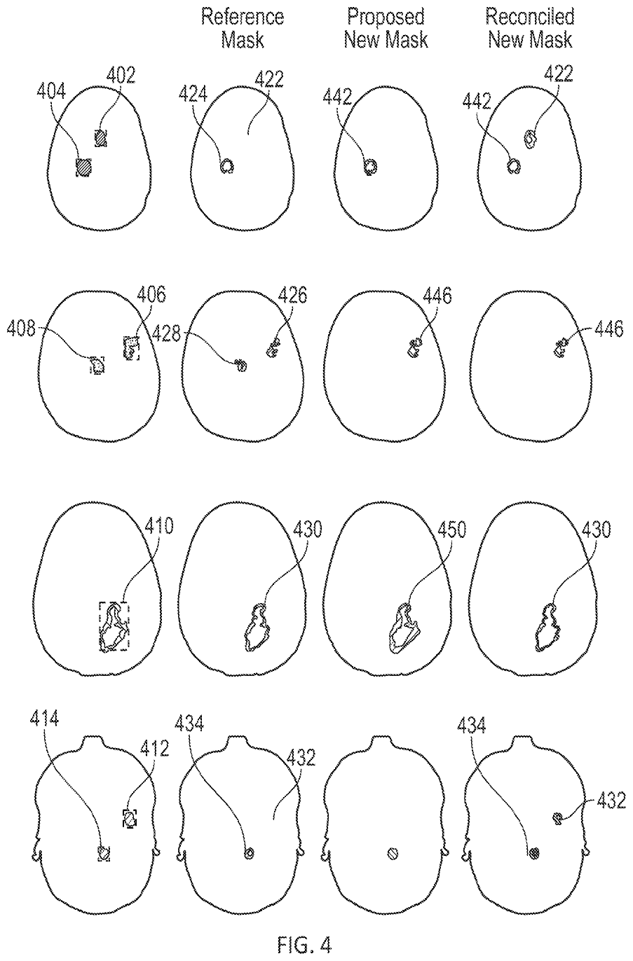

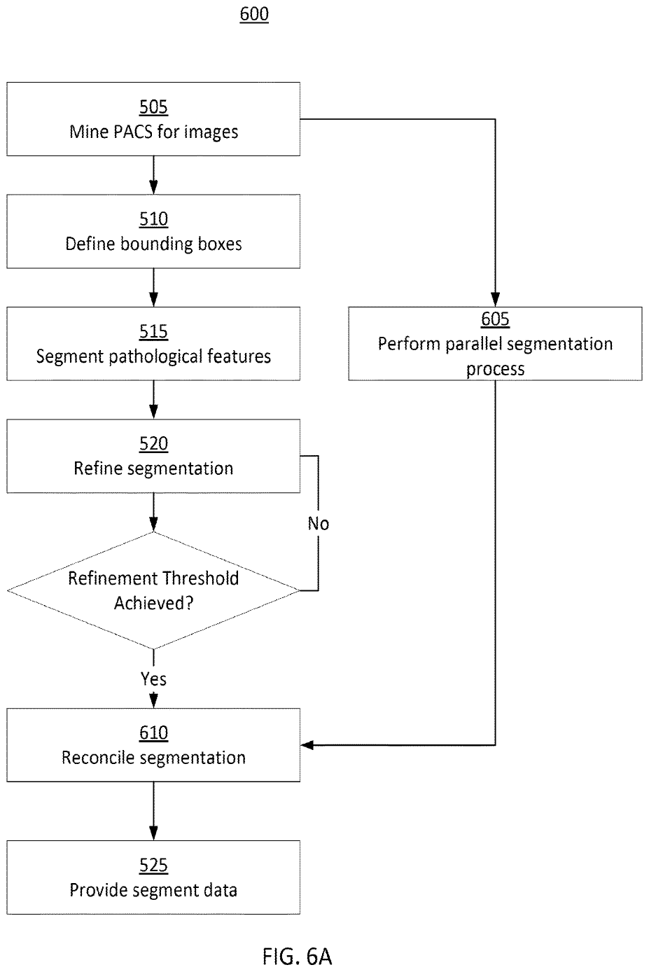

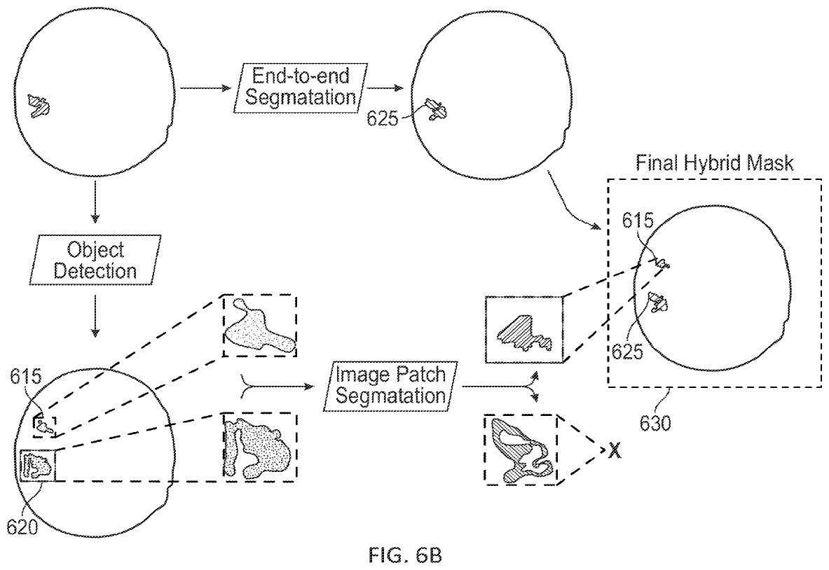

Referring to FIG. 4, reconciliation between various segmentations is depicted, according to some embodiments. For example, a first bounding box 402 and second bounding box 404 are disposed over an image. A first reference mask 422 and a second reference mask 424 (corresponding to the first bounding box 402 and second bounding box 404, respectively) are depicted. A proposed new mask (which may be generated by a self-refinement process, as further discussed with regard to FIG. 3, a parallel process, as is further discussed with regard to FIG. 6, or another process) does not identify a feature corresponding to at least one bounding box. For example, a mask corresponding to the first bounding box may not be identified. A proposed mask 442 corresponding to the second bounding box can be retained in a reconciled image, along with the first reference mask 422.

A third bounding box 406 and a fourth bounding box 408 are disposed over another image. A respective third reference mask 426 and a fourth reference mask 428 can be defined; the third reference mask 426 can be refined to generate a third proposed mask 446, in a new mask image. The refinement can degrade or omit the pathological feature described by the fourth reference mask 428. The third proposed mask 446 and fourth reference mask 428 can be included in a reconciled image. A fifth bounding box 410 disposed over another image can have a fifth reference mask 430 defined and associated therewith. A fifth proposed mask 450 may be detected but fail an additional criterion such as a volume or pixel count threshold, thus the reconciled image includes the fifth reference mask 430 rather than the fifth proposed mask 450. A further image includes a sixth bounding box 412 and seventh bounding box 414, which are associated with a respective sixth reference mask 432 and seventh reference mask 434. No viable proposed masks are included in another image (e.g., a refined iteration, or another mask determined based on end-to-end segmentation). Thus, a reconciled image includes the sixth reference mask 432 and seventh reference mask 434. For example, the reconciled image can be identical to a reference image.

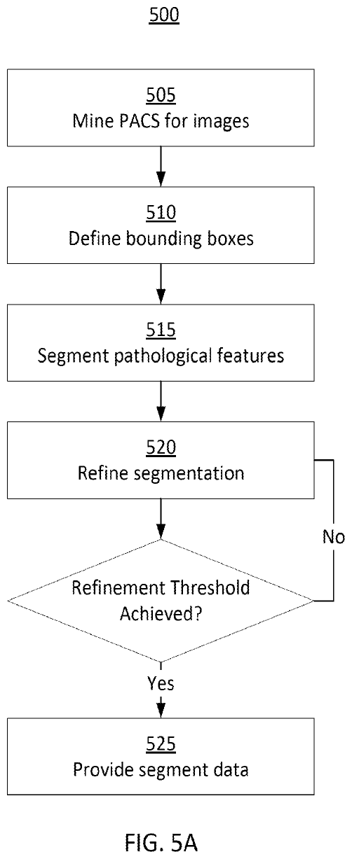

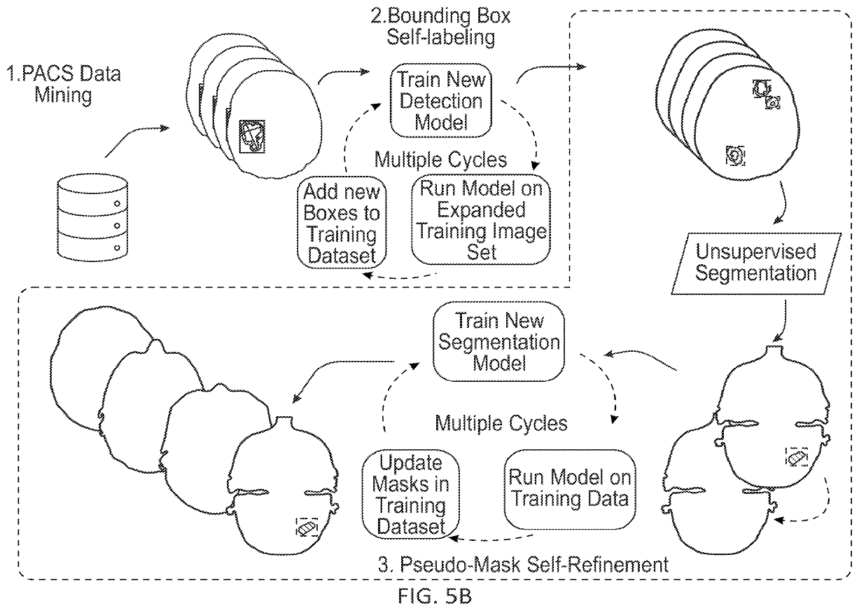

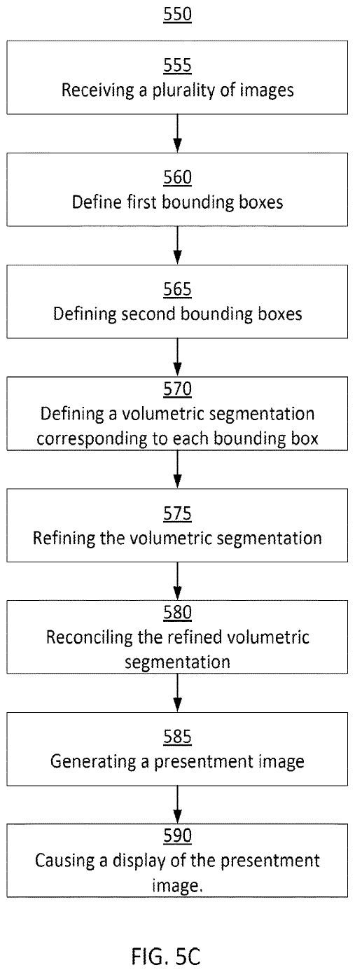

FIG. 5A depicts a method 500 of segmenting pathological features, according to some embodiments. The method 500 can be performed by one or more components or systems described in FIG. 7 or throughout this disclosure. In brief summary, at operation 505 PACS is mined for images and corresponding line annotations. At operation 510, line annotations are converted to bounding boxes and the bounding box training is performed, defining bounding boxes for any pathological features lacking data mined annotations. Pathological features of the bounding boxes are defined (e.g., by segmentation/masks) at operation 515. The segmentations/masks are refined at operation 520. The segmentation/mask data is provided at operation 525.

Still referring to FIG. 5A, and with further reference to operation 505, PACS is mined for images and corresponding line annotation data. The images can be mined based on metadata (e.g., tags) associated therewith (e.g., a pathological condition, patient name, file type, associated department, and the like). The images can be mined based on a characteristic or type of the image. For example, a CT scan can be associated with tuberculosis, lung cancer, or another plurality of pathological conditions; brain MRIs can be associated with brain tumors, indications of stroke, or other pathological conditions. In some embodiments, the data mining can include a detection of pathological features in an image. For example, one or more components of a data mining system can discriminate between two pathological conditions of an image (e.g., between a tumor and a broken bone in an x-ray). In some embodiments, the mining process can be supervised e.g., a plurality of mined images can be displayed, such as by a file name or pictographically on a graphical user interface to enable supervision of the detection of the mined images. A portion of mined images (e.g., according to a confidence threshold) can require approval for inclusion in the plurality of mined image. In some embodiments, the mining can include a weighted score wherein various data (e.g., those disclosed herein) are assigned weights which can be combined to assign a score to an image, which can determine if the image is included in the training set, presented to a user for approval, rejected, or otherwise processed. In some embodiments, the mining (and indeed, each operation described herein) can be performed automatically, without human intervention. Alternatively or in addition, line annotation data not available for mining from PACS may be separately introduced by the loading of tabular data files (e.g., .csv or .xlsx).

In some embodiments, the training data can be selected according to patient information. For example, one or more of the images can be mined according to an age, sex, comorbidity, weight, or other patient attribute. In some embodiments, the images can be selected as a continuous learning system. For example, responsive to the defined bounding boxes, or operations disclosed herein (e.g., segmentation operations), an iterative set of images can be mined. For example, a bounding box, segmentation, or the like can indicate the presence of a pathological feature abutting an organ, bone, or other feature; additional images can be mined based on the location of pathological features proximal to the organ, bone, or other feature. Alternatively or in addition, newly acquired images containing annotations (e.g. line, arrow, segmentation, and/or polyline annotations) can be mined from PACS and introduced into the system, resulting in a newly enlarged training dataset.

At operation 510, bounding boxes are defined. A portion of the bounding boxes can be defined by a conversion of the line annotations received, determined, or defined at operation 505. For example, the line annotations can be converted to bounding boxes by the line to bounding box converter 102. In some embodiments, the bounding box training component can train a model based on the bounding boxes of the bounding box converter 102, and the model may be refined or employed by the bounding box refinement component. The bounding boxes can be defined by a model trained based on the line annotations included in the mined images, and can define bounding boxes on additional images lacking such line annotations, or other images, such as one or more images of interest that the trained model is applied to detect bounding boxes for. A portion of the bounding boxes can be defined for lesions having line annotations associated therewith. The bounding boxes defined based on the line annotations may be referred to as converted from the line annotations. Such conversion includes a (e.g., rectangular) bounding box overlaying a dimension defined by the line annotations such that each terminus of a line annotation is disposed along the bounding box boundary. However, such a conversion is not intended to be limiting. A conversion can include any bounding box defined based on the line annotations. For example, a bounding box can be defined having one or more greater, lesser, or different dimensions than a line annotation the bounding box is converted from, or a same or different orientation, so long as the bounding box is converted (i.e., defined) based on the line annotations. For example, a line annotation can be converted to a bounding box by a model trained, designed, or operational to detect the line annotations or a pathological feature (e.g., may be placed based on both the line annotations and the pathological feature).

A further portion of the bounding boxes can be defined for lesions lacking line annotations. The bounding boxes can surround all or a portion (e.g., lobe, section, or the like) of an image or pathological feature thereof. Some bounding boxes may be adjacent, overlapping, or otherwise be associated. A linkage or other association between respective bounding boxes can be established or a bounding box can be defined atomically, without regard to additional bounding boxes. In some embodiments, bounding boxes can be disposed on a plurality of images. For example, a plurality of slices of an MRI image, or a plurality of longitudinal MRI images of a patient. The additional images can be bounded to illustrate changes therebetween, or to further train a model for the application of bounding boxes, or other operations herein disclosed.

At operation 515, pathological lesions (also referred to herein as features) corresponding to the bounding boxes are defined. For example, the pathological lesions can be defined for one or more images of interest or images mined at operation 505. Each bounding box can include one or more pathological feature; each pathological feature can be included in one or more bounding boxes. The definition of the feature can include defining portions thereof extending beyond, between or within a bounding box. The definition of the feature can include defining segmentations of the pathological feature. The segmentations can be illustrated according by a mask overlaying the image. For example, the pathological features corresponding to the bounding boxes can be defined according to the techniques discussed herein and variations thereof, such as with respect to the masks of FIG. 3.

At operation 520, the segmentations are refined. The refinement can be of any type, such as according to various smoothing, sharpening, or other techniques. The refinement can be based on a machine learning model trained based on a plurality of segmentations. For example, the segmentations can be or include manual segmentations, or automated segmentations based on the defined pathological features at operation 515. The refinements can be for the various data mined images, or for the one or more images of interest. In some embodiments, the refinement process or other processes herein can include iterative steps. For example, operations 505, 510, and 515 can be repeated based on predefined inputs, or based on refinements of segmentations of pathological features performed at operation 520. Indeed, various operations herein can be repeated, modified, omitted, or substituted. For example, the segmentations can be refined according to any of the techniques discussed herein and variations thereof, such as with respect to the refinement cycles of FIG. 3. Refinement can include bound checking or other reconciliation. For example, refined segmentations can be reconciled relative to previously refined or defined images (e.g., to avoid loss or degradation of a previously detected pathological feature). For example, reconciliation can be performed according to any of the techniques discussed herein and variations thereof, such as with respect to the reconciliations of FIG. 4. In some embodiments, a plurality of refinement cycles can be performed. For example, a refinement threshold can be evaluated for each iteration. The operation can complete upon reaching the threshold.

At operation 525, segmentation data is provided. For example, the segmentation data can be provided by a graphical user interface (e.g., for presentment to a user), or to another computing system or component thereof. For example, the segmentation data can be passed to a volumetric analysis component which can determine various parameters of the segmentation, such as a volumetric burden of a pathological feature, a surface area, or other characterization based on size, shape, roughness, or proximity to another feature of interest (e.g., an artery or brainstem). In some embodiments, one or more parameters can be presented to a user on a graphical user interface. For example, any of a line annotation, a bounding box, a segmentation, or the like can be presented to a user. The line annotation, bounding box, segmentation, or the like can be presented independently or overlaid on an image and can include additional parametric data, such as a volume thereof. In some embodiments, the presentment can include information from a plurality of images, for example, a presentment can include a 2D or 3D volume based on a plurality of images, a recession or expansion based on a series of longitudinal images, etc. In some embodiments, the segmentation data can be presented to a database for additional processing or access. For example, a machine learning model can be trained based on the segmentation data. The provision of data can include a prognosis, treatment recommendation, an association or linkage to a peer group of patients, etc. FIG. 5B is a pictographic depiction of the method 500 of FIG. 5A.

FIG. 5C is a variation method 550 of the method 500 of FIG. 5A. The disclosed variation method 550 can be performed by various processors coupled to at least one non-transitory memory. For example, the variation method 550 can be performed by one or more components or systems described in FIG. 7 or throughout this disclosure. In brief summary, a plurality of images are received at operation 555. At operation 560, a detection model is trained. At operation 565, bounding boxes are defined. At operation 570, volumetric segmentations corresponding to each bounding box are defined. At operation 575, the volumetric segmentations are refined. At operation 580, the refined volumetric segmentations are reconciled. At operation 585, a presentment image is generated. At operation 590, a display of the presentment image is caused.

Still referring to FIG. 5C, and with further reference to operation 555, a plurality of images are received. At least a portion of the images can include line annotations depicting a dimension of one or more pathological features of the images. For example, the plurality of images can be or include images mined from a PACS system (e.g., as described with regard to operation 505 of method 500), a curated image set, or any other plurality of images wherein at least a portion thereof includes line annotations dimensioning a pathological feature. Alternatively or in addition, separately curated bounding boxes (e.g., from a .csv file) can be introduced directly in operation 565. The received images can include one or more further images of interest. For example, the images of interest can be a diagnostic image of a patient. The image of interest, and the other plurality of images can be received concurrently, or at various times. For example, additional images can be received to train or retrain various models throughout the operation. The image of interest can be received prior to the other plurality of images (e.g., to select the plurality of images based on the image of interest), subsequent to receipt of the plurality of interest (e.g., to allow pre-training of a dataset), or interspersed with the receipt of additional images. At operation 560, a detection model is trained, based on the line annotations or the pathological features. For example, the line to bounding box converter can be trained to define bounding boxes surrounding the pathological features of the received images. The training can be based on identification of the line annotations (e.g., on a portion of the images including line annotations), or an identification of the pathological features themselves (e.g., on a portion of the images including line annotations or lacking line annotations), their effect on adjoining tissues, or the like. In some embodiments, the training can be of a machine learning model using the received images as a training set.

At operation 565, bounding boxes are defined. For example, the bounding boxes can be defined as described with regard to operation 505. The bounding boxes can be defined over the received plurality of images, such as all images, or one or more images of interest. At operation 570, a volumetric segmentation (e.g., a mask) is defined for at least one bounding box of at least one image (e.g., one or more images of interest). The segmentation can be based on a trained model, or other technique. For example, as is referred to with regard to operation 515 of method 500, Otsu's method, can be employed to define a volumetric segmentation corresponding to each bounding box. At operation 575, the volumetric segmentations can be refined based on volumetric segmentation training data. For example, the volumetric segmentation training data can include a previously reconciled segmentation (e.g., reconciled segmentations included or excluded from a subsequent mask). For example, the volumetric segmentations can be refined according to operation 520 of the method 500 of FIG. 5A. At operation 580, the refined volumetric segmentations are reconciled. For example, the refined segmentations can be reconciled with another volumetric segmentation corresponding to the same pathological feature. In some embodiments the reconciliation can be performed as described by operation 520 of the method of FIG. 5a. In some embodiments, operation 580, or other operations of the variation method 550 can be omitted, substituted, modified, etc., in light of any aspect of the present disclosure.

At operation 585, a presentment image is generated. The presentment image can include an image of interest. The image of interest can be overlaid with additional information. For example, the image can be overlaid with information as is described with regard to operation 525. At operation 590, the display of a presentment of the image is caused. For example, causing the image to be displayed can include outputting a signal to a display such as a diagnostics image monitor, a computer monitor, or another display. The display can be a display of the system of FIG. 7 or be interfaced thereto. For example, the data can be conveyed over various media such as a network for display at a monitor disposed remote from one or more components of the system (e.g., the one or more processors). The presentment can be or include all or a portion of an image. For example, the presentment can include a presentment of an area of interest such as one or more pathological features of the image. The display can be caused by interfacing with another component or system to display the presentment image. For example, the system can provide the presentment image over a network (e.g., the internet) to a system designed, configured, or operational to depict the image. For example, the image can be conveyed to a terminal or other device of a patient or medical practitioner for presentment therefrom.

FIG. 6A depicts a method 600 of segmenting pathological features, according to some embodiments. The method 600 can be performed by one or more components or systems described in FIG. 7 or throughout this disclosure. In brief summary, at operation 505 PACS is mined for images. At operation 510, bounding boxes are defined. Pathological features of the bounding boxes are defined at operation 515. The segmentations are refined at operation 520. A parallel segmentation process is performed at operation 605. The segmentations are reconciled at operation 610. The segmentation data is provided at operation 525.

Operations 505, 510, 515, 520, and 525 may be performed as described with reference to the method 500 of FIG. 5, with various substitutions, omissions, and additions as are disclosed herein, and are not repeated merely for the sake of brevity. At operation 605, a parallel segmentation process is performed. The parallel segmentation process can include one or more determinations of segmentations of a pathological feature. For example, the parallel segmentation process can be a different process, such as an end-to-end unsupervised process to determine a segmentation of one or more pathological features. In some embodiments, a plurality of parallel segmentation processes can be performed. For example, a plurality of CNN or other models can be used to determine one or more segmentations of a pathological feature. The parallel segmentation process can repeat, omit, substitute, or modify one or more operations of the method 500 of FIG. 5. For example, the parallel segmentation process can process the same data mined images (e.g., as a training set), and one or more images of interest as method 500 of FIG. 5, or can include a different training set, such as from a different PACS, or a different group (e.g., based on selection criteria or a random selection thereof). The parallel segmentation process may be performed at a same or different resolution, bit depth, or other parameter thereof.

At operation 610, the segmentations are reconciled. For example, segmentations defined by the one or more parallel processes are reconciled between and with any segmentations of the one or more segmentations defined at operation 520. For example, reconciliation can be performed according to any of the techniques discussed herein and variations thereof, such as with respect to the reconciliations of FIG. 4. For example, images can be reconciled on an image basis, a bounding box basis, or another basis. For example, segmentations generated by an end-to-end process can be associated with the bounding boxes defined at operation 510, such as based on a location on an image. The segmentations generated according to one or more parallel operations (e.g., an end-to-end operation) can be preferred, such that a viable segmentation can be accepted based on the generating process. The segmentations (e.g., segmentations associated with respective bounding boxes) can be selected according to one or more parameters, such as contiguity, conformance to another image, etc. For example, a generating process of a segmentation or various parameters can be weighted and a highest weighted segmentation can be selected for inclusion in a reconciled image.

FIG. 6B is a pictographic depiction of the method of FIG. 6A. For example, the pictographic depiction includes a first segmentation 615 and a second segmentation 620 defined at operation 515, defining a first pathological feature and a second pathological feature, respectively. A third segmentation 625, defining the second pathological feature is defined according to operation 605. The various segmentations are reconciled to form a final hybrid mask 630. For example, the third segmentation 625 of the hybrid mask 630 can be associated with (e.g., linked to) the second segmentation 620 based on a similar location thereof. The third segmentation 625 can be selected for inclusion in the final hybrid mask 630 based on the end-to-end process, and the first segmentation 615 can be selected for inclusion in the final hybrid mask 630 based on the lack of a corresponding viable detected feature in the parallel process. The nomenclature of the final hybrid mask 630 merely refers to depiction thereof, and does not preclude further processing or refinement operations thereto. For example, the final hybrid mask 630 can be further refined or input to further refine one or more models.

### B. Computing and Network Environment

Various operations described herein can be implemented on computer systems. FIG. 7 shows a simplified block diagram of a representative server system 700, client computer system 714, and network 726 usable to implement certain embodiments of the present disclosure. In various embodiments, server system 700 or similar systems can implement services or servers described herein or portions thereof. Client computer system 714 or similar systems can implement clients described herein. The system 100 described herein can be similar to the server system 700. Server system 700 can have a modular design that incorporates a number of modules 702 (e.g., blades in a blade server embodiment); while two modules 702 are shown, any number can be provided. Each module 702 can include processing unit(s) 704 and local storage 706.

Processing unit(s) 704 can include a single processor, which can have one or more cores, or multiple processors. In some embodiments, processing unit(s) 704 can include a general-purpose primary processor as well as one or more special-purpose co-processors such as graphics processors, digital signal processors, or the like. In some embodiments, some or all processing units 704 can be implemented using customized circuits, such as application specific integrated circuits (ASICs) or field programmable gate arrays (FPGAs). In some embodiments, such integrated circuits execute instructions that are stored on the circuit itself. In other embodiments, processing unit(s) 704 can execute instructions stored in local storage 706. Any type of processors in any combination can be included in processing unit(s) 704.

Local storage 706 can include volatile storage media (e.g., DRAM, SRAM, SDRAM, or the like) and/or non-volatile storage media (e.g., magnetic or optical disk, flash memory, or the like). Storage media incorporated in local storage 706 can be fixed, removable or upgradeable as desired. Local storage 706 can be physically or logically divided into various subunits such as a system memory, a read-only memory (ROM), and a permanent storage device. The system memory can be a read-and-write memory device or a volatile read-and-write memory, such as dynamic random-access memory. The system memory can store some or all of the instructions and data that processing unit(s) 704 need at runtime. The ROM can store static data and instructions that are needed by processing unit(s) 704. The permanent storage device can be a non-volatile read-and-write memory device that can store instructions and data even when module 702 is powered down. The term “storage medium” as used herein includes any medium in which data can be stored indefinitely (subject to overwriting, electrical disturbance, power loss, or the like) and does not include carrier waves and transitory electronic signals propagating wirelessly or over wired connections.

In some embodiments, local storage 706 can store one or more software programs to be executed by processing unit(s) 704, such as an operating system and/or programs implementing various server functions such as functions of the system 100 of FIG. 1 or any other system described herein, or any other server(s) associated with system 100 or any other system described herein.

“Software” refers generally to sequences of instructions that, when executed by processing unit(s) 704 cause server system 700 (or portions thereof) to perform various operations, thus defining one or more specific machine embodiments that execute and perform the operations of the software programs. The instructions can be stored as firmware residing in read-only memory and/or program code stored in non-volatile storage media that can be read into volatile working memory for execution by processing unit(s) 704. Software can be implemented as a single program or a collection of separate programs or program modules that interact as desired. From local storage 706 (or non-local storage described below), processing unit(s) 704 can retrieve program instructions to execute and data to process in order to execute various operations described above.

In some server systems 700, multiple modules 702 can be interconnected via a bus or other interconnect 708, forming a local area network that supports communication between modules 702 and other components of server system 700. Interconnect 708 can be implemented using various technologies including server racks, hubs, routers, etc.

A wide area network (WAN) interface 710 can provide data communication capability between the local area network (interconnect 708) and the network 726, such as the Internet. Technologies can be used, including wired (e.g., Ethernet, IEEE 802.3 standards) and/or wireless technologies (e.g., Wi-Fi, IEEE 802.11 standards).

In some embodiments, local storage 706 is intended to provide working memory for processing unit(s) 704, providing fast access to programs and/or data to be processed while reducing traffic on interconnect 708. Storage for larger quantities of data can be provided on the local area network by one or more mass storage subsystems 712 that can be connected to interconnect 708. Mass storage subsystem 712 can be based on magnetic, optical, semiconductor, or other data storage media. Direct attached storage, storage area networks, network-attached storage, and the like can be used. Any data stores or other collections of data described herein as being produced, consumed, or maintained by a service or server can be stored in mass storage subsystem 712. In some embodiments, additional data storage resources may be accessible via WAN interface 710 (potentially with increased latency).

Server system 700 can operate in response to requests received via WAN interface 710. For example, one of modules 702 can implement a supervisory function and assign discrete tasks to other modules 702 in response to received requests. Work allocation techniques can be used. As requests are processed, results can be returned to the requester via WAN interface 710. Such operation can generally be automated. Further, in some embodiments, WAN interface 710 can connect multiple server systems 700 to each other, providing scalable systems capable of managing high volumes of activity. Other techniques for managing server systems and server farms (collections of server systems that cooperate) can be used, including dynamic resource allocation and reallocation.

Server system 700 can interact with various user-owned or user-operated devices via a wide-area network such as the Internet. An example of a user-operated device is shown in FIG. 7 as client computing system 714. Client computing system 714 can be implemented, for example, as a consumer device such as a smartphone, other mobile phone, tablet computer, wearable computing device (e.g., smart watch, eyeglasses), desktop computer, laptop computer, and so on.

For example, client computing system 714 can communicate via WAN interface 710. Client computing system 714 can include computer components such as processing unit(s) 716, storage device 718, network interface 720, user input device 722, and user output device 724. Client computing system 714 can be a computing device implemented in a variety of form factors, such as a desktop computer, laptop computer, tablet computer, smartphone, other mobile computing device, wearable computing device, or the like.

Processor 716 and storage device 718 can be similar to processing unit(s) 704 and local storage 706 described above. Suitable devices can be selected based on the demands to be placed on client computing system 714; for example, client computing system 714 can be implemented as a “thin” client with limited processing capability or as a high-powered computing device. Client computing system 714 can be provisioned with program code executable by processing unit(s) 716 to enable various interactions with server system 700.

Network interface 720 can provide a connection to the network 726, such as a wide area network (e.g., the Internet) to which WAN interface 710 of server system 700 is also connected. In various embodiments, network interface 720 can include a wired interface (e.g., Ethernet) and/or a wireless interface implementing various RF data communication standards such as Wi-Fi, Bluetooth, or cellular data network standards (e.g., 3G, 4G, LTE, etc.).

User input device 722 can include any device (or devices) via which a user can provide signals to client computing system 714; client computing system 714 can interpret the signals as indicative of particular user requests or information. In various embodiments, user input device 722 can include any or all of a keyboard, touch pad, touch screen, mouse or other pointing device, scroll wheel, click wheel, dial, button, switch, keypad, microphone, and so on.

User output device 724 can include any device via which client computing system 714 can provide information to a user. For example, user output device 724 can include a display to display images generated by or delivered to client computing system 714. The display can incorporate various image generation technologies, e.g., a liquid crystal display (LCD), light-emitting diode (LED) including organic light-emitting diodes (OLED), projection system, cathode ray tube (CRT), or the like, together with supporting electronics (e.g., digital-to-analog or analog-to-digital converters, signal processors, or the like). Some embodiments can include a device such as a touchscreen that functions as both input and output device. In some embodiments, other user output devices 724 can be provided in addition to or instead of a display. Examples include indicator lights, speakers, tactile “display” devices, printers, and so on.

Some embodiments include electronic components, such as microprocessors, storage and memory that store computer program instructions in a computer readable storage medium. Many of the features described in this specification can be implemented as processes that are specified as a set of program instructions encoded on a computer readable storage medium. When these program instructions are executed by one or more processing units, they cause the processing unit(s) to perform various operations indicated in the program instructions. Examples of program instructions or computer code include machine code, such as is produced by a compiler, and files including higher-level code that are executed by a computer, an electronic component, or a microprocessor using an interpreter. Through suitable programming, processing unit(s) 704 and 716 can provide various functionality for server system 700 and client computing system 714, including any of the functionality described herein as being performed by a server or client, or other functionality.

It will be appreciated that server system 700 and client computing system 714 are illustrative and that variations and modifications are possible. Computer systems used in connection with embodiments of the present disclosure can have other capabilities not specifically described here. Further, while server system 700 and client computing system 714 are described with reference to particular blocks, it is to be understood that these blocks are defined for convenience of description and are not intended to imply a particular physical arrangement of component parts. For instance, different blocks can be but need not be located in the same facility, in the same server rack, or on the same motherboard. Further, the blocks need not correspond to physically distinct components. Blocks can be configured to perform various operations, e.g., by programming a processor or providing appropriate control circuitry, and various blocks might or might not be reconfigurable depending on how the initial configuration is obtained. Embodiments of the present disclosure can be realized in a variety of apparatus including electronic devices implemented using any combination of circuitry and software.

### C. Example Embodiment

The example method is directed to tumor segmentation which may, for example, provide clinical value by more accurately quantifying tumor burden than conventional line measurements, such as for determining patient prognosis or aiding radiomic analysis. While fully supervised learning can yield high-performing segmentation models, the time and effort required to manually label large training sets may limit some applications. More particularly, the present example is directed to data mined line annotations to facilitate the development of a brain MRI tumor segmentation models without the need for manually segmented training data.

A tumor detection model trained using clinical line annotations mined from PACS can be leveraged with unsupervised segmentation (Otsu thresholding) to generate pseudo-masks of enhancing tumors on T1-weighted post-contrast images. Baseline segmentation models can be trained using a model such as a CNN (e.g., U-Net, Mask R-CNN, or HRNet architectures) employed within a semi-supervised learning (SSL) framework to automatically refine the pseudo-masks. Following each self-refinement cycle, a new model can be trained and tested on a held-out set consisting of manually segmented images, with the SSL cycles continuing until Dice score coefficient (DSC) peaks. Model DSCs can be compared using bootstrap resampling.

Two example implementation methods are disclosed: (1) end-to-end segmentation, and (2) a hybrid pipeline augmenting end-to-end segmentation with detection plus image patch segmentation. According to at least some data, the hybrid pipeline may outperform end-to-end segmentation alone. Thus line annotations mined from PACS can be harnessed using automated techniques to produce accurate brain MRI tumor segmentation models without manually segmented training data, providing a potential mechanism to rapidly establish tumor segmentation capabilities across radiology modalities.

The application of artificial intelligence (AI) to diagnostic radiology promises to revolutionize patient care by reducing detection errors, increasing accuracy, and improving the non-invasive characterization of disease. In cancer imaging, tumor segmentation can represent a more accurate quantification of disease burden as compared with the conventional linear measurements used by in routine care and oncology clinical trials. In contrast to tumor segmentation, linear measurements, which assume that tumors have a regular ellipsoid morphology, are relatively insensitive to subtle changes in tumor size and may be impacted by technical factors like patient positioning on the scanner, which may reduce interobserver agreement. Tumor segmentation can be a useful (or prerequisite) step for at least some methods or operations of radiomic analysis, the application of AI for the characterization of lesions such as tumors to guide optimal treatment, presenting a barrier to “big data” cancer investigations involving radiology image data.

While AI has transformed the automated processing of non-medical images, the progress of radiology AI has been comparatively slow due to the scarcity of domain experts needed for the time-intensive and generally non-reimbursable process of radiology image labeling. Segmentation of a single tumor on a brain MRI can take an average of ten minutes, and one volumetric scan may feature numerous individual lesions. Robust segmentation models typically require many annotated training images, and deployed models may require additional annotated data for retraining due to limitations of model generalizability and target data shift according to at least some desired performance levels for some data. The reliance on manual annotation of radiology data for fully supervised learning thus represents a formidable challenge and major obstacle to the advancement of radiology AI. Scalable automated image annotation techniques that avoid large-scale manual efforts, shifting the major burden of effort from the radiologist to the AI models and data scientists, may be useful.

An automated pipeline can data mine clinically-generated tumor line measurement annotations in PACS for use within a semi-supervised learning (SSL) framework for tumor detection on brain MRI. In achieving accurate automated tumor detection using noisy image annotations generated in well-established clinical radiology workflows, this pipeline can avoid a manual annotation bottleneck associated with some fully supervised learning approaches and enable access to an existing source of continuous annotation data for model retraining.

These data mined line annotations and the resultant tumor detection capability can be extended to facilitate further downstream AI tasks such as tumor segmentation, accelerating the development of models that will improve the accuracy of disease response assessment and enable scalable “big data” radiomic analysis. More particularly, there remains a need for semantic segmentation methods that achieve high performance without manual training set curation, instead using noisy image annotations drawn from real world clinical radiology workflows. In the current example, we disclose systems and methods for data mining and tumor detection development pipeline using fully automated AI methodologies to achieve accurate segmentation of enhancing tumors on brain MRI, such as without the need for manually segmented training data, or with a reduced set thereof with reference to at least some alternatives.

The tumor detection model can be trained on T1-weighted post-contrast brain MR images from various patients. The training pipeline data can mine line annotations from PACS, convert lines to bounding boxes, and utilize bounding boxes within a semi-supervised framework to automatically correct unlabeled tumors in the training images. A model trained with the expanded training set can achieve relatively high accuracy, such as an F1 score of 0.954 in detecting tumors ≥1 cm on a held-out test set.

In the current example, this tumor detection model can be utilized to automatically generate baseline tumor segmentation pseudo-masks as follows. The model can be used to detect lesions on the detection training image set, yielding lesion bounding boxes on images. Within the image patch defined by each bounding box, Otsu thresholding, a method of automatically binarizing an image into separate foreground and background masks using histogram intensity analysis, can be applied to generate an initial approximation of the enhancing tumor. The resultant set of images and segmentation pseudo-masks comprised the initial dataset used to train the baseline tumor segmentation models.

To establish the reference standard test dataset used to score each segmentation model, images containing enhancing tumors from patients (e.g., adult patients) can be randomly selected from the previously described data mined held out test cohort. Manual segmentation of all enhancing tumors can be performed in ITK-SNAP (such as by an experienced radiologist). To avoid data leakage, patient overlap between the training and test datasets can be avoided.

End-to-end (full image) segmentation model training and pseudo-mask self-refinement: various segmentation neural network architectures can be employed. For example, U-Net, Mask R-CNN, and HRNet. All models can be measured for average Dice score coefficient (DSC) using the held-out test set. To improve the baseline segmentation pseudo-masks through the addition of contextual image data, an automated self-refinement process utilizing the full brain MR images and masks can be employed for each architecture, as follows.

Operation 1: The baseline pseudo-mask training set (Train_{Otsu}) can be employed to train a baseline segmentation model (Model_{Otsu}), which can be used to predict masks on the training images themselves, generating a new proposed segmentation mask set. An automated acceptability check, (e.g., designed to prevent the erosion and eventual loss of a detected lesion's segmentation mask during self-refinement), can be performed on each image's proposed new mask, leveraging the bounding boxes previously generated by the detection model. This can yield a new segmentation mask training set, Train₁.

Operation 2: The new training set (generically, Trainₙ) can be used to train a new model (Modelₙ), which can be again used to predict new masks on the training images. The acceptability check can be performed for each image and proposed mask, generating a new segmentation task training set, Trainₙ₊₁.

Operation 2 can be repeated until model performance, as scored with the held-out test set, peaks. The training set yielding the overall best-performing trained model was selected as the final optimized segmentation dataset (Train_{Final}) and used to train final models of each architecture.

To investigate whether the tumor detection model has value beyond generating segmentation training data (e.g., to directly augment the end-to-end model's performance), a second hybrid segmentation inference pipeline is disclosed. The hybrid pipeline can include a second arm running in parallel to the end-to-end (full image) segmentation model. Use of the detection model can be used to identify tumors and explicitly serve up image patches to a separate image patch segmentation model. The predicted masks output by each arm of the pipeline can be combined in a final reconciliation step to correct for tumors “missed” by the end-to-end segmentation model and maximize overall segmentation performance.

To generate the image patch segmentation training set, the training images and Train_{Final} segmentation masks can be cropped using the bounding boxes provided by the detection model. The cropped images and masks (Train_{Final-bbox}) can be used to train an image patch segmentation model utilizing each architecture.

Statistical analysis: For each model, DSC 95% confidence intervals can be calculated by bootstrap resampling (e.g., 2000 times). Each resampling cycle, a sample of images can be randomly selected from the full test dataset with replacement and used to score each model. These aggregate results can be used to calculate pair-wise model comparison p-values (1-sided).

For an end-to-end (e.g., full image) segmentation model, the baseline segmentation models can be trained using the Otsu-generated segmentation pseudo-masks (e.g., to achieve DSC of 0.768 (U-Net), 0.831 (Mask R-CNN), or 0.838 (HRNet)). The automated self-refinement method can significantly improve performance for each architecture. For example, performance can peak within 10 cycles (e.g., U-Net in 6 cycles (p<0.001); 0.871, Mask R-CNN in 6 cycles (p<0.001); 0.873, HRNet in 7 cycles (p<0.001)). Representative examples of segmentation mask evolution using the self-refinement process are included in FIG. 3.

The self-refined segmentation mask dataset may yield the best-performing trained model (e.g., HRNet after 7 cycles) can be selected as the Train_{Final} dataset used to train the segmentation models of each architecture (e.g., attaining maximum DSC of 0.809 (U-Net), 0.871 (Mask R-CNN), and 0.873 (HRNet)).

Image patch segmentation model and hybrid pipeline models can be trained using the Train_{Final-bbox} dataset. Some data may achieve DSC of 0.737 (U-Net), 0.801 (Mask R-CNN), or 0.796 (HRNet). For each architecture, the hybrid pipeline may improve performance over the final (end-to-end) segmentation model alone: DSC 0.832 (U-Net), 0.884 (Mask R-CNN), and 0.881 (HRNet). Comparing end-to-end and hybrid segmentation using the best-performing architectures for each approach, the hybrid pipeline may significantly outperform end-to-end segmentation alone: maximum DSC 0.884 (Mask R-CNN) vs. 0.873 (HRNet), p <0.001.

In this investigation, use of a brain MRI tumor detection model trained using line annotations mined from PACS can achieve accurate automated segmentation of enhancing tumors without manually segmented training data. The use of an unsupervised segmentation method, Otsu thresholding, in concert with the detection model output can generate a baseline segmentation training set that is automatically improved using an SSL self-refinement process. In providing contextual brain MRI information in self-refinement, models iteratively improved at delineating tumor from non-tumor tissue despite very noisy initial pseudo-masks, yielding significantly better performing final end-to-end segmentation models. Augmenting these end-to-end segmentation models with a parallel arm consisting of tumor detection followed by image patch segmentation may further improve performance, achieving DSC of 0.884 (p<0.001).

In considering the larger context of the PACS mining, object detection, and segmentation (MODS) development pipeline extended by this investigation, there are several important advantages over large-scale manual segmentation efforts for traditional fully supervised learning. Foremost, the automated generation and self-refinement of segmentation training data may be markedly faster, shifting the major annotation burden to the models themselves and limiting the manual annotation required by radiologists to only the comparatively small test set. In one embodiment, manual segmentation of the test set required approximately 20 hours of total effort (3.8 minutes per image), which was accomplished by one radiologist over 7 sessions. At the same per-image rate, manual annotation of the training set itself (n=9,702 images) would have required over 610 hours (more than 25 full days) of additional radiologist effort, a >95% time savings afforded by using the MODS approach. Second, since the MODS approach is scalable, increasing the size of the segmentation training set (e.g., by an order of magnitude) could be accomplished without any additional manual effort from radiologists. This addresses a critical need for pixel-level image annotation methods that can be feasibly applied to massive data sets to achieve high statistical power in “big data” multimodal radiomic investigations. Third, MODS is lightweight in its modular design, enabling the dynamic rerunning of the full development pipeline to incorporate novel unsupervised segmentation methods or address target data shifts (e.g., due to changes in patient population, scanner, or imaging protocol). Whereas in a fully supervised learning framework a data shift occurring in late-stage development or post-deployment typically requires major manual effort to rework training annotations, the MODS pipeline may interface with existing radiology workflows. This may aid reruns using newly mined image and line annotation data, supporting radiology AI's transition from static deep learning models to dynamic pipelines and continuous learning.

There are several potential limitations to accomplishing automated tumor segmentation using the MODS approach, such as limitations specific to the provided example, which may be obviated by other variations otherwise disclosed herein. First, by design, this method may achieve segmentation by relying on the previous tumor detection capability. While this can facilitate overall pipeline optimization by reducing the opaque, “black box” nature of a single end-to-end segmentation model, various additional limitations to the detection model may degrade the performance of the segmentation model. Inadequate segmentation performance due to deficiencies inherited from the detection model may be countered through the use of a retraining segmentation dataset for segmentation model fine-tuning. Second, while the application of Otsu thresholding to generate the baseline pseudo-masks was successful for enhancing tumors on brain MRI, achieving a high DSC of 0.838 (HRNet) using only these noisy, unrefined training data, this technique may achieve a lower DSC for tumors or imaging modalities with low lesion conspicuity, potentially resulting in baseline pseudo-masks and segmentation models that derive differing benefit from self-refinement. Thus, various thresholding or segmentation methods may be employed. Further, other unsupervised segmentation techniques could be investigated and formally compared using a held-out test set to quantify the various performances thereof across one or more data sets. Third, the optimal deployment of the hybrid segmentation pipeline within a continuous learning framework may require modifications to radiology information technology architectures to support data routing, clinical workflow integration, and model re-training.

Thus, our example demonstrates the value of historical line annotation data mined from PACS for facilitating radiology computer vision model development. Applying automated AI techniques to these mined weak annotations yields tumor detection and segmentation models achieving excellent performance, reducing the manual annotation time and effort required of radiologists as compared with fully supervised learning. The MODS development pipeline could be applied to other radiology imaging modalities, providing a roadmap to rapidly establish and continuously optimize automated tumor detection and segmentation capabilities across the radiology department.

Although various embodiments are contemplated, some non-limiting implementations options are provided henceforth. Segmentation models can be trained using the PyTorch framework. All images can be normalized to intensity values (e.g., from 0-1) or resized (e.g., to 512×512 pixels). Training duration can range from 15 to 20 epochs per model. Learning rate can be determined automatically at train time using the learning rate finder function in FastAI and can be adjusted (e.g., every 3 epochs).

Following one self-refinement cycle, each proposed new segmentation mask can undergo an acceptability check, as follows. For each of the image's detection bounding boxes, the intersection of the proposed new segmentation mask and bounding box area (“sub-mask”) can be calculated. If the sub-mask is less than a defined threshold (e.g., ≤35%) of the bounding box area, the new proposed sub-mask was rejected, preserving the previous cycle's sub-mask (i.e., no update is made to this region of the full image mask or the full image thereof). Otherwise, the new proposed sub-mask can be accepted, replacing the previous cycle's sub-mask. For all regions of the image outside of the detection bounding boxes, the proposed new sub-mask can be accepted automatically.

For the hybrid segmentation pipeline reconciliation, the predicted masks output by each parallel branch in the hybrid pipeline can be combined in a final reconciliation step to increase the performance of each arm, (e.g., as depicted in FIG. 4). For each of the image's detection bounding boxes, the intersection of the full image segmentation model's predicted mask and bounding box area (“sub-mask”) can be calculated. If the sub-mask is less than a defined threshold (e.g., ≤35%) of the bounding box area, the sub-mask can be rejected and the image patch segmentation model's output was utilized as the sub-mask for that region of the image. Otherwise, the sub-mask can be accepted, and the image patch segmentation model output for that region of the image can be discarded. For one or more regions of the image outside of the detection bounding boxes, any segmentation sub-masks predicted by the full image segmentation model can be accepted automatically.

Although various variations can occur among populations, pathological conditions, and other variables, the disclosed example can be most closely associated with the following populations. The systems and methods described herein are not intended to be limited thereby; indeed, the systems and methods described herein can be applied to various groups, pathological conditions, image data, and other variables, as well as random variation therebetween.

| | Training group | Testing group | Total |
|---|---|---|---|
| Patient demographics | | | |
| No. of patients | 3,449 | 93 | 3,542 |
| Mean age (y) | 58.5 ± 17.0 | 56.4 ± 17.0 | 58.4 ± 17.0 |
| Women/men | 1,964/1,484 | 49/45 (1.11) | 2,013/1,529 |
| (ratio) | (1.32) | | (1.32) |
| Primary malignancy | | | |
| Lung | 466 (13.5%) | 11 (11.8%) | 477 (13.5%) |
| adenocarcinoma | | | |
| Glioblastoma | 367 (10.6%) | 12 (12.9%) | 379 (10.7%) |
| Breast ductal | 342 (9.9%) | 9 (9.6%) | 351 (9.9%) |
| carcinoma | | | |
| Melanoma | 131 (3.8%) | 10 (10.8%) | 141 (4.0%) |
| Prostate | 87 (2.5%) | 1 (1.1%) | 88 (2.5%) |
| adenocarcinoma | | | |
| Lung small cell | 71 (2.1%) | 5 (5.4%) | 76 (2.1%) |
| carcinoma | | | |
| Other, multiple, | 2,072 (60.1%) | 46 (49.5%) | 2,148 (60.6%) |
| or unavailable | | | |
| Number of unique | 6,236 | 229 | 6,465 |
| scans | | | |
| Image slice thickness | | | |
| (mm) | | | |
| 5.0 | 9,496 (95.8%) | 316 (99.1%) | 9,812 (95.9%) |
| 3.0 | 257 (2.6%) | 3 (0.9%) | 260 (2.5%) |
| 4.5 | 98 (1.0%) | N/A | 98 (1.0%) |
| Other | 60 (0.6%) | N/A | 60 (0.6%) |
| Mean bounding box | 2.18 ± 1.23 | 1.95 ± 1.21 | 2.17 ± 1.23 |
| length (cm) | | | |

Although elsewhere referred to herein, example DSC are consolidated and provided henceforth, according to the present example. The Number of refinement cycles can vary according to various patient populations, variations of systems or methods, and other modifications. Some such modifications are disclosed herein explicitly; others will be understood by one skilled in the art, in view of the present disclosure.

| | U-Net | Mask R-CNN | HRNet |
|---|---|---|---|
| | DSC | | |
| Baseline Otsu- | 0.768 | 0.838 | 0.838 |
| generated pseudo- | | | |
| masks | | | |
| Self-refinement | | | |
| cycle # | | | |
| 1 | 0.777 | 0.859 | 0.852 |
| 2 | 0.779 | 0.861 | 0.865 |
| 3 | 0.784 | 0.868 | 0.866 |
| 4 | 0.791 | 0.869 | 0.869 |
| 5 | 0.793 | 0.869 | 0.872 |
| 6 | 0.798 | 0.871 | 0.872 |
| 7 | 0.797 | 0.867 | 0.873 |
| 8 | 0.794 | 0.862 | 0.869 |
| 9 | 0.792 | 0.862 | 0.868 |

While the disclosure has been described with respect to specific embodiments, one skilled in the art will recognize that numerous modifications are possible. Embodiments of the disclosure can be realized using a variety of computer systems and communication technologies including, but not limited, to specific examples described herein. Embodiments of the present disclosure can be realized using any combination of dedicated components and/or programmable processors and/or other programmable devices. The various processes described herein can be implemented on the same processor or different processors in any combination. Where components are described as being configured to perform certain operations, such configuration can be accomplished, e.g., by designing electronic circuits to perform the operation, by programming programmable electronic circuits (such as microprocessors) to perform the operation, or any combination thereof. Further, while the embodiments described above may make reference to specific hardware and software components, those skilled in the art will appreciate that different combinations of hardware and/or software components may also be used and that particular operations described as being implemented in hardware might also be implemented in software or vice versa.

Computer programs incorporating various features of the present disclosure may be encoded and stored on various computer readable storage media; suitable media include magnetic disk or tape, optical storage media such as compact disk (CD) or DVD (digital versatile disk), flash memory, and other non-transitory media. Computer readable media encoded with the program code may be packaged with a compatible electronic device, or the program code may be provided separately from electronic devices (e.g., via Internet download or as a separately packaged computer-readable storage medium).

Thus, although the disclosure has been described with respect to specific embodiments, it will be appreciated that the disclosure is intended to cover all modifications and equivalents within the scope of the following claims.

Aspects can be combined and it will be readily appreciated that features described in the context of one aspect can be combined with other aspects. Aspects can be implemented in any convenient form. For example, by appropriate computer programs, which may be carried on appropriate carrier media (computer readable media), which may be tangible carrier media (e.g., disks) or intangible carrier media (e.g., communications signals). Aspects may also be implemented using a suitable apparatus, which can take the form of one or more programmable computers running computer programs arranged to implement the aspect. As used in the specification and in the claims, the singular form of “a”, “an”, and “the” include plural referents unless the context clearly dictates otherwise. References to “or” may be construed as inclusive so that any terms described using “or” may indicate any of a single, more than one, and all of the described terms.

What is claimed is:

1. A system for automated segmentation of pathological lesions, the system comprising:
one or more processors coupled to a non-transitory memory, the one or more processors configured to:
receive a plurality of first images, a first portion of the plurality of first images comprising line annotations corresponding to one or more pathological lesions;
convert a first plurality of line annotations to a first plurality of corresponding bounding boxes;
train a lesion detection model to generate a second plurality of bounding boxes, based on the first plurality of bounding boxes;
generate, based on the lesion detection model, a third bounding box for one or more unannotated lesions of a second image;
define a volumetric segmentation corresponding to the third bounding box;
refine the volumetric segmentation based on volumetric segmentation training data to define a refined volumetric segmentation;
reconcile the refined volumetric segmentation with another volumetric description corresponding to at least one of the one or more unannotated lesions;
generate a presentment image, based on the reconciliation; and
cause a display of the presentment image via a graphical user interface.

2. The system of claim 1, wherein the one or more processors are configured to:
determine a parallel volumetric segmentation; and
reconcile the refined volumetric segmentation with the parallel volumetric segmentation to define the presentment image.

3. The system of claim 1, wherein at least one of the one or more unannotated lesions comprise a tumor and the second image is one or more slices of a magnetic resonance imaging (MRI) image.

4. The system of claim 1, wherein at least one of the one or more unannotated lesions is lung cancer and the second image is a computed topography (CT) scan.

5. The system of claim 1, wherein the plurality of first images includes a second portion of the plurality of first images lacking the line annotations of the one or more pathological lesions.

6. The system of claim 1, wherein:
the second image comprises a plurality of two-dimensional slices of a three-dimensional structure; and
the volumetric segmentation is a three-dimensional volumetric segmentation.

7. The system of claim 1, wherein, to reconcile the refined volumetric segmentation, the one or more processors are configured to iteratively refine the volumetric segmentation based on the volumetric segmentation training data, each iterative refinement comprising a generation of an iterative segmentation of the one or more pathological lesions.

8. A method for segmenting pathological lesions, the method comprising:
receiving, by one or more processors coupled to a non-transitory memory, a plurality of first images, a first portion of the plurality of first images comprising line annotations corresponding to one or more first pathological lesions;
defining, by the one or more processors, a plurality of first bounding boxes converted from the line annotations of the plurality of first images;
defining, by the one or more processors, a second bounding box for one or more second pathological lesions of a second image using a detection model trained from the plurality of first bounding boxes;
defining, by the one or more processors, a volumetric segmentation corresponding to each bounding box of the second image;
refining, by the one or more processors, the volumetric segmentation based on volumetric segmentation training data to define a refined volumetric segmentation;
reconciling, by the one or more processors, the refined volumetric segmentation with another volumetric segmentation corresponding to at least one of the one or more second pathological lesions;
generating, by the one or more processors, a presentment image, based on the reconciliation; and
causing, by the one or more processors, a display of the presentment image via a graphical user interface.

9. The method of claim 8, comprising:
determining, by the one or more processors, a parallel volumetric segmentation; and
reconciling, by the one or more processors, the refined volumetric segmentation with the parallel volumetric segmentation to define the presentment image.

10. The method of claim 8 wherein at least one of the one or more second pathological lesions comprise a tumor and the second image is one or more slices of a magnetic resonance imaging (MRI) image.

11. The method of claim 8 wherein at least one of the one or more second pathological lesions is lung cancer and the second image is a computed topography (CT) scan.

12. The method of claim 8 wherein the plurality of first images includes a second portion of the plurality of first images lacking the line annotations of the one or more first pathological lesions.

13. The method of claim 8, wherein:
the second image comprises a plurality of two-dimensional slices of a three-dimensional structure; and
the volumetric segmentation is a three dimensional volumetric segmentation.

14. The method of claim 8, wherein, to reconcile the refined volumetric segmentation, the one or more processors are configured to:
iteratively refine the volumetric segmentation based on the volumetric segmentation training data, each iterative refinement comprising a generation of an iterative segmentation of the one or more first pathological lesions.

15. A non-transitory computer-readable medium having instructions that, upon execution by a computing device, cause the computing device to perform operations comprising:
receiving an image comprising a pathological lesion;
ingesting, by a model trained to detect lesions based on a marking, the image;
generating, based on the model trained to detect lesions, a marking for one or more unannotated lesions of the image;
defining a volumetric segmentation corresponding to the marking;
refining the volumetric segmentation based on volumetric segmentation training data to define a refined volumetric segmentation;
reconciling the refined volumetric segmentation with another volumetric description corresponding to the one or more unannotated lesions;
generating a presentment image, based on the reconciliation; and
causing a display of the presentment image via a graphical user interface.

16. The computer-readable medium of claim 15, wherein the marking comprises at least one of:
a bounding box; or
a line annotation.

17. The computer-readable medium of claim 15, comprising instructions to cause the computing device to perform operations comprising:
determining a parallel volumetric segmentation; and
reconciling the refined volumetric segmentation with the parallel volumetric segmentation to define the presentment image.

18. The computer-readable medium of claim 15, wherein at least one of the one or more unannotated lesions comprise a tumor and the image is one or more slices of a magnetic resonance imaging (MRI) image.

19. The computer-readable medium of claim 15, wherein:
the image comprises a plurality of two-dimensional slices of a three-dimensional structure; and
the volumetric segmentation is a three dimensional volumetric segmentation.

20. The computer-readable medium of claim 15, wherein the instructions to refine the volumetric segmentation comprise instructions that, upon execution by the computing device, cause the computing device to perform operations comprising:
iteratively refining the volumetric segmentation based on the volumetric segmentation training data, each iterative refinement comprising a generation of an iterative segmentation of the pathological lesion.
